(19) United States
(12) Patent Application Publication
(10) Pub. No.: US 2012/0065536 A1
(43) Pub. Date: Mar. 15, 2012

(54) SYSTEM AND METHOD FOR NEUROLOGICAL EVALUATION

(76) Inventors: Elvir CAUSEVIC, Clayton, MO (US); Miroslav Sazdovski, Sarajevo (BA); Anel Hadziaganovic, Sarajevo (BA); Haris Lacevic, Sarajevo (BA)

(21) Appl. No.: 12/879,287

(22) Filed: Sep. 10, 2010

(51) Int. Cl. A61B 5/0476

(52) U.S. Cl. 600/544

(57) ABSTRACT

A device and method for acquiring and processing a patient's brain electrical activity is provided. Noise contamination during acquisition and transmission of the brain electrical signals is reduced by providing differential amplifiers in the patient sensor in close proximity to the electrodes. A guarding technique is applied in the patient sensor to avoid inductive coupling of low frequency environmental noise. Radio-frequency (RF) filters and a Faraday cage assembly are also provided in the patient sensor to reduce electromagnetic interference. The brain electrical signals acquired by the electrodes are transmitted through shielded cables to a handheld base unit for signal processing.

The present disclosure generally relates to a medical apparatus, and more particularly, to a method and system for acquiring and processing brain electrical signals using a portable neurological assessment device.

All of the brain's activities, whether sensory, cognitive, emotional, autonomic, or motor function, is electrical or biochemical in nature. Through a series of electro-chemical reactions, mediated by molecules called neurotransmitters, electrical potentials are generated and transmitted throughout the brain, traveling continuously between and among the myriad of neurons. This activity establishes the basic electrical signatures of the electroencephalogram (EEG) and creates identifiable frequencies which have a basis in anatomic structure and function. Understanding these basic rhythms and their significance makes it possible to characterize the brain electrical signals as being within or beyond normal limits. At this basic level, the electrical signals serve as a signature for both normal and abnormal brain function. Just as an abnormal electrocardiogram (ECG) pattern is a strong indication of particular heart pathology, an irregular brain wave pattern is a strong indication of particular brain pathology.

Traditional brain wave recording systems measure electrical potentials between electrodes placed on the scalp of a patient and generate a record of the electrical activity of the brain. Typically, such electrical activity is shown as a set of analog waveforms or signals that is analyzed by an EEG technician and then presented to a neurologist for interpretation and clinical assessment. This process can be time-consuming, expensive, technically demanding and subject to human error. Since the results are not rapidly available, the traditional systems for analyzing brain electrical activity are not well suited for use in emergency rooms or other point-of-care settings.

Currently, emergency room patients with altered mental status, acute neuropathy, or head trauma must undergo costly and time-consuming brain imaging studies that visualize the structure of the brain, for example computed tomography (CT) and magnetic resonance imaging (MRI). Unfortunately, in many cases, the clinical condition of patients can continue to deteriorate as they wait for equipment to become available or for specialists to interpret the scans. Further, many functional brain abnormalities which eventually have anatomical and structural consequences are often not visible, or take time to become visible on a CT scan or MRI. For example, intoxication, concussion, active seizure, metabolic encephalopathy, infections, diabetic coma and numerous other conditions show no abnormality on CT scan. A classical stroke, or a traumatic brain injury (TBI), may not be immediately visualized by an imaging test even if there is a clear and noticeably abnormal brain function. Similarly, diffuse axonal injury (DAI), related to shearing of nerve fibers which is present in majority of concussive brain injury cases, can remain invisible on most routine structural images. If undetected at an early stage, swelling or edema from DAI can subsequently lead to coma and death. This indicates the need for real-time, functional brain state assessment technology, which can be performed in the ER, or in an ambulatory setting, and can detect emergency neurological conditions hours ahead of the standard clinical assessment tools available today. Similarly, there is a need for a field-portable assessment tool for detection of TBI in soldiers out in the battlefield, and also for detection of sports related brain injury in athletes. Rapid, on-the-field assessments may help prevent repeat injuries and “second impact syndrome” in soldiers and athletes already suffering from a first traumatic brain impact.

Functional brain state assessment can be made by recording and analyzing brain electrical activity of patients with reported neurological injury. Handheld, easy-to-administer brain wave assessment devices would facilitate neurological evaluation of patients at the point-of-care, which in turn would allow rapid and proper initiation of therapy. Presently, portable brain wave recording systems measure a patient's brain electrical impulses and transmit the acquired signals to an external signal processing device for signal analysis and data processing. Such systems may additionally perform other steps in the external signal processing module, including signal amplification, artifact rejection, classification of signal features and display one or more classification results. Exemplary systems for point-of-care neuro-assessment are disclosed in commonly assigned U.S. application Ser. Nos. 11/195,001, 12/041,106, 12/059,014 and 12/639,357, which are incorporated herein by reference in their entirety. Such systems generally feature a headset with an array of electrodes configured to detect and transmit raw brain electrical signals to an external processing device. Such systems may also record raw evoked potentials following administration of a stimulus. The acquired signals are transmitted to an external device for processing in order to limit the power, space and weight constraints on the headset. The external device processes data using various signal processing methods, including traditional Fast Fourier Transform (FFT) analysis, wavelet transforms and Linear Discriminant Analysis. Alternate signal processing tools, such as diffusion geometric analysis, have also been used advantageously in the analysis of brain electrical activity, as disclosed in commonly assigned U.S. application Ser. No. 12/105,439, which is also incorporated herein by reference in its entirety.

However, the complexity and/or inefficiencies in current signal acquisition and transmission methodologies lower the quality of the acquired brain electrical signals. For example, environmental noises tend to degrade the acquired brain electrical signals during transmission of the signals to an external processing device. Transmission of signals through unshielded or partially shielded cables to an external device exposes the signals to inductive coupling of low frequency environmental noise, and electromagnetic interference (EMI) due to radiated or conducted RF (radio frequency) emissions. On the other hand, shielded cables introduce environmental low frequency noise by creating parasitic capacitances between the signal leads and the grounded shield as well as inter-lead capacitances causing degradation of the common mode rejection ratio (CMRR). The noise contamination degrades the quality of the acquired brain electrical signals and limits the applicability of neurological evaluation devices. The present disclosure provides methods and systems for providing noise protection to the brain electrical signals during signal acquisition and transmission to increase signal strength, maintain signal integrity, reduce common mode noise, and provide a high level of confidence in signal transmissions.

The present disclosure provides methods and systems for reducing noise contamination during acquisition and transmission of analog bioelectric signals, adaptable amplification of the signals, and specifically the reduction of common noise interference. Although the methods and systems are described here with reference to the acquisition, transmission and processing of brain electrical activity (EEG signals), they can be applied for the acquisition and transmission of any bioelectric signals. Advantages of the present invention may include, but are not limited to, reducing noise contamination, enabling the acquisition and processing of sub-microvolt signals, facilitating device integration, reducing common mode noise, and reducing complexity of system deployment.

In one aspect of the present disclosure, a device for acquiring and processing brain electrical signals from a patient is provided. The device comprises a patient sensor and a handheld base operatively coupled to the patient sensor for processing the brain electrical signals. The patient sensor comprises at least one active electrode and at least one reference electrode to be attached to the patient's head, and at least one differential amplifier receiving input signals from the at least one active electrode and the at least one reference electrode. Additionally, the patient sensor comprises two-stage radio frequency (RF) filters comprising a feed-through RF capacitor and a symmetrical common mode filter. In some exemplary embodiments, the patient sensor further comprises a modified Driven Right Leg (DRL) circuit to provide a guard signal for the electrode common, and a Faraday cage assembly to provide continuous RF shielding to the circuitry in the patient sensor.

In another aspect of the present disclosure, a method of reducing noise contamination during acquisition of bioelectric signals from a patient is provided. The method comprises the steps of providing a patient sensor comprising at least one active electrode channel, at least one reference electrode channel and at least one differential amplifier receiving inputs from the active and reference electrodes. The method further comprises the steps of providing two-stage RF filters, a DRL circuit to generate a guard signal, and a Faraday cage assembly in the patient sensor to provide noise protection. Differential measurements of the bioelectric signals are made using the at least one differential amplifier. In exemplary embodiments, the output signals of the amplifier are transmitted through a shielded cable to a handheld base unit for further processing.

Additional embodiments consistent with principles of the invention are set forth in the detailed description which follows or may be learned by practice of methods or use of systems or articles of manufacture disclosed herein. It is understood that both the foregoing general description and the following detailed description are exemplary and explanatory only, and are not restrictive of the invention as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention. In the drawings:

FIG. 1 is a block diagram of an exemplary embodiment of a neuro-assessment apparatus for acquiring and processing brain electrical signals;

FIG. 2 shows a schematic circuitry of an exemplary patient sensor of the neuro-assessment apparatus of the present disclosure;

FIG. 3 illustrates a Driven Right Leg circuit of an exemplary patient sensor of the neuro-assessment apparatus of the present disclosure;

FIG. 4 illustrates AC current switches for measuring electrode impedance, as may be included in an exemplary patient sensor of the neuro-assessment apparatus of the present disclosure;

FIG. 5 illustrates the MOSFET DC current sources for checking electrode impedance/resistance, as may be included in an exemplary patient sensor of the neuro-assessment apparatus of the present disclosure;

FIG. 6 shows the power supply circuitry of an exemplary patient sensor of the neuro-assessment apparatus of the present disclosure;

FIGS. 7A and 7B show brain electrical signals transmitted using standard electrode cables and an exemplary embodiment of the patient sensor, respectively;

FIGS. 8A and 8B show brain electrical signals that were exposed to 900 MHz electromagnetic signal and transmitted using standard electrode cables and an exemplary embodiment of the patient sensor, respectively;

FIGS. 9A and 9B show brain electrical signals that were exposed to 1800 MHz electromagnetic signal and transmitted using standard electrode cables and an exemplary embodiment of the patient sensor, respectively.

### DESCRIPTION OF ILLUSTRATIVE EMBODIMENTS

Reference is now made in detail to exemplary embodiments of the present disclosure, examples of which are illustrated in the accompanying drawings. Wherever possible, the same reference numbers will be used throughout the drawings to refer to the same or like parts. The embodiments are described in sufficient detail to enable one skilled in the art to practice and use the invention, and it is to be understood that other embodiments may be utilized and that electrical, logical, and structural changes may be made without departing from the spirit and scope of the present disclosure.

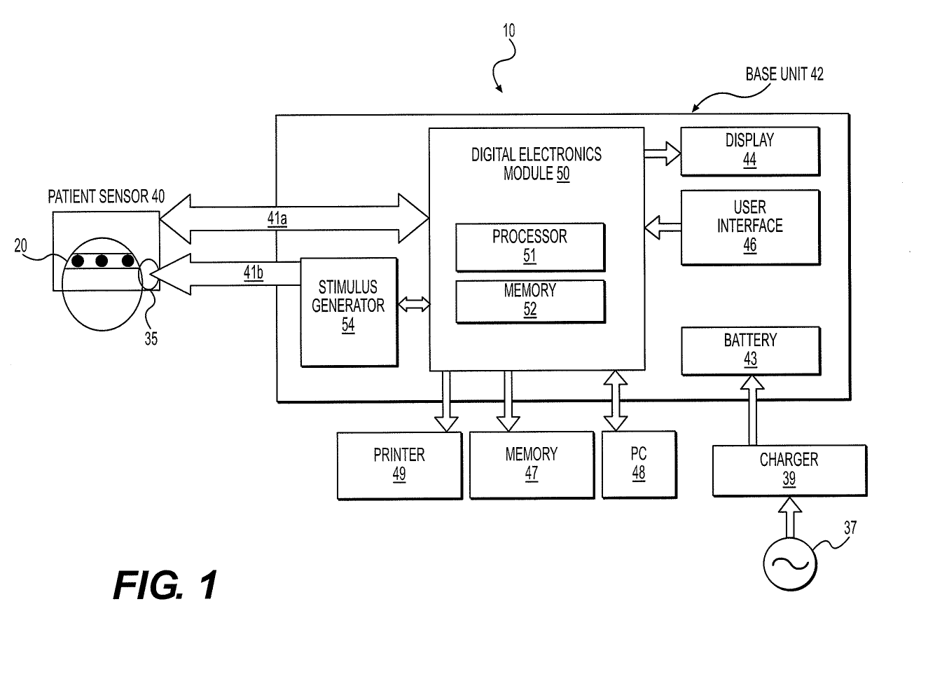

In an exemplary embodiment, data corresponding to brain electrical activity is used to detect neurological injury or disease in patients. The brain electrical signals are measured and analyzed at the point-of-care using a portable neuro-assessment device. In accordance with embodiments consistent with the present disclosure, FIG. 1 shows a neuro-assessment apparatus 10 for acquiring and processing brain electrical signals, and providing an evaluation of the patient's neurological condition. In an exemplary embodiment, the neuro-assessment apparatus 10 is implemented as a portable device for point-of-care applications. The apparatus consists of a patient sensor 40 which may be coupled to a base unit 42, which can be handheld, as illustrated in FIG. 1.

In some exemplary embodiments, patient sensor 40 includes an electrode array 20 comprising one or more disposable neurological electrodes to be attached to a patient's head to acquire brain electrical signals. Alternately, in some embodiments, individual electrodes may be provided. The electrodes are configured for sensing both spontaneous brain activity as well as evoked potentials generated in response to applied stimuli. In one exemplary embodiment, electrode array 20 comprises anterior (frontal) electrodes: F1, F2, F7, F8, Fz′ (also referred to as AFz) and Fpz (ground electrode) to be attached to a patient's forehead, and electrodes A1 and A2 to be placed on the front or back side of the ear lobes, or on the mastoids, in accordance with the International 10/20 electrode placement system (with the exception of Fz′). Alternate placements or “modified 10/20” electrode arrangements can also be used. Because of the noise reduction and signal amplification capability of the present invention, it is possible to place electrodes closer together (thus generating less potential), than is possible using the standard 10/20 system. The use of a limited number of electrodes enable rapid and repeatable placement of the electrodes on a patient, which in turn facilitates efficient, and more accurate, patient monitoring. In one illustrative embodiment, the electrodes are positioned on a low-cost, disposable platform, which can serve as a “one-size-fits-all” sensor. For example, electrodes 20 may be mounted on a headgear that is configured for easy and/or rapid placement on a patient. In another embodiment, electrodes 20 are freely arranged on the head of the patient without the use of a headgear. Free electrode placement facilitates free selection of the reference electrode for differential measurements and allows different montage configurations. Other electrode configurations may be utilized as and when required, as would be understood by those of ordinary skill in the art.

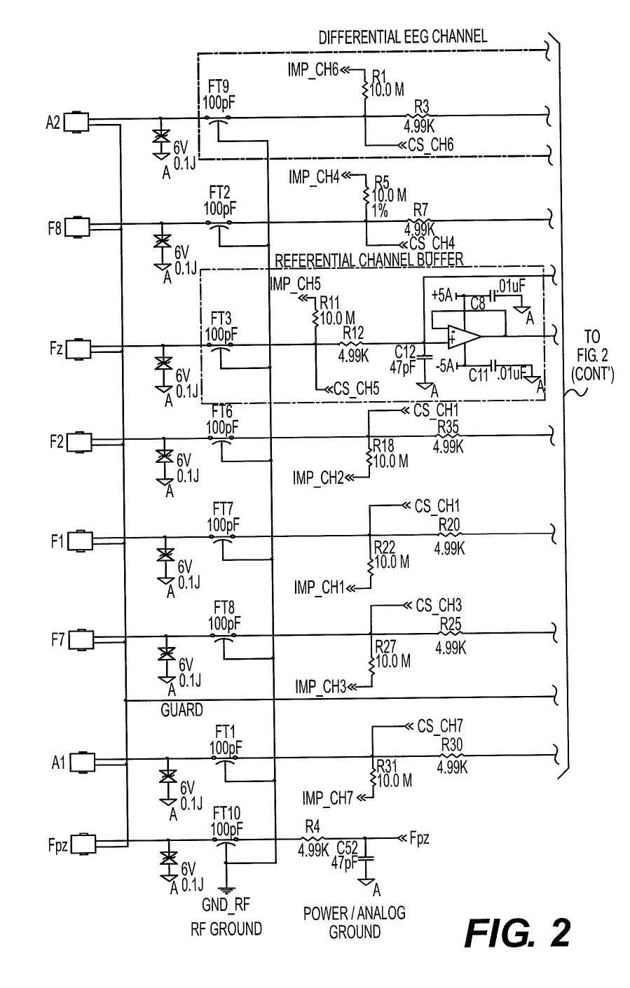

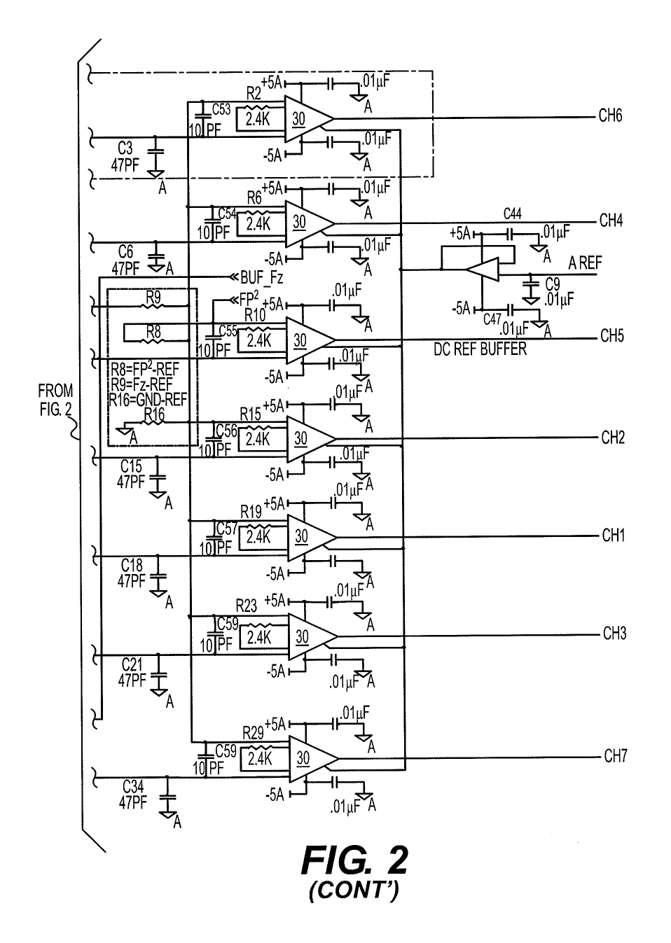

Patient sensor 40 further comprises a pre-amplification circuit, which includes one or more analog electrode channels CH1, CH2, . . . , CHn. The pre-amplification circuit is positioned in close proximity to the one or more disposable neurological electrodes. In certain embodiments, the pre-amplification circuit is positioned within 15 cm of the neurological electrodes. FIG. 2 shows an exemplary schematic of patient sensor 40 comprising seven electrode channels (CH1, CH2, . . . , CH7) corresponding to eight electrodes (F1, F2, F7, F8, Fz′, Fpz, A1 and A2). Differential measurements are made between electrode pairs using high input impedance instrumentation amplifiers 30. Instrumentation amplifiers are designed to amplify signal difference and reject input signals common to both input leads. This is important when recording brain electrical signals, because each electrode acquires different brain potential, but noise influence is similar on both electrodes channels due to their close proximity. Therefore, instrumentation amplifiers 30 reject the noise that is common to both the input leads of the amplifiers. Particular care must be taken in the physical layout of the pre-amplification circuit, such that the conductors in the printed circuit are of the same or similar length and shape, so as to minimize differences in common mode. In exemplary embodiments, a referential montage is used for the differential measurements. As shown in FIG. 2, three different signals-Fz′, Fpz or analog/power ground signal—can be used as the common reference for the differential measurements. In an exemplary embodiment, if Fz′ signal is used as reference, the zero-ohm R9 resistor (Fz′-REF) is implemented and the R8 (Fpz-REF) and R16 (GND-REF) resistors are omitted. Similarly, if Fpz is used as the reference, the zero-ohm R8 resistor is implemented while R9 and R16 are omitted. If the analog/power ground is used as reference, the zero-ohm resistor R16 is implemented while R9 and R8 are omitted). In some exemplary embodiments, precision instrumentation amplifiers, such as AD8221BR, (hereinafter referred to as “IA”) are used for the differential measurements. IA provides very high CMRR (Common Mode Rejection Ratio) over the entire EEG frequency range, assuming proper circuit layout techniques are followed. In addition to the extreme DC input resistance (in the order of 1G Ohm), IA have very low input common mode and differential capacitance of around 2 pF. This ensures the high system CMRR of more than 105 dB within the EEG frequency spectrum. Additional features of the exemplary IA include very low input noise of 8 nV/√Hz at 1 kHz and maximum input voltage noise of 0.25 μV p-p (0.1 Hz to 10 Hz). This ensures that the device noise floor in the example is at a maximum of 2 μV p-p within the EEG frequency range. The instrumentation amplifiers 30 act as impedance translators (high-to-low). By having instrumentation amplifiers close to the electrodes 20 in the patient sensor 40, low impedance signals, which are less sensitive to noise, are transmitted to the base unit 42. As a result, the transmitted brain electrical signals become less susceptible to parasitic capacitances between the signal leads and the grounded shield as well as inter-lead capacitances.

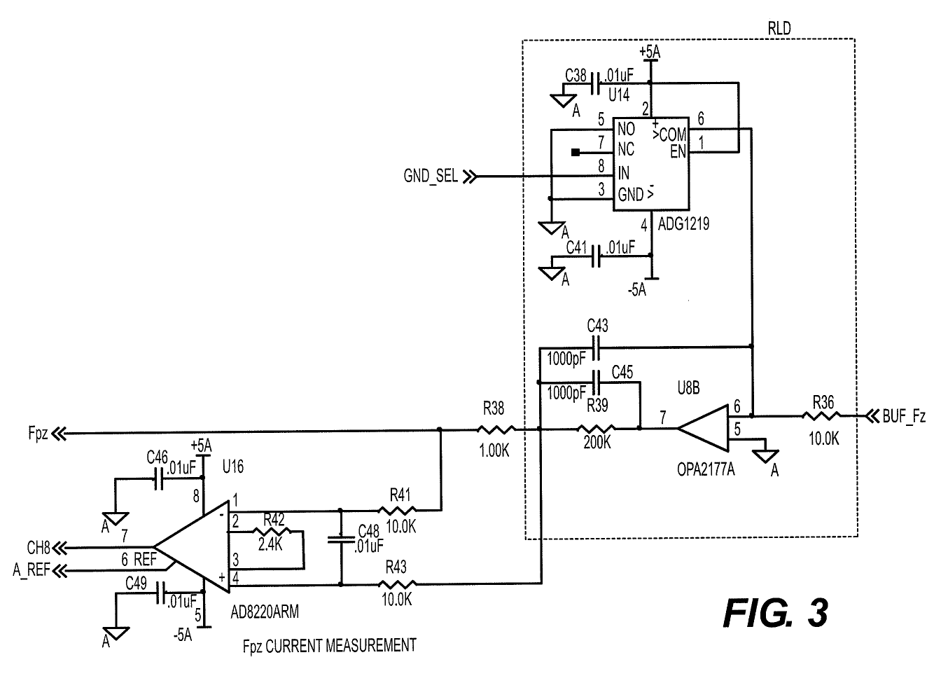

In some exemplary embodiments, additional circuitry is used together with the instrumentation amplifiers to aid in the common mode noise rejection. In certain embodiments, a Driven Right Leg Circuit or “DRL” circuit, modified for use with neurological electrodes, is added to the pre-amplifier circuit to reduce common mode interference. The DRL circuit is used to eliminate interference noise by actively canceling out the interference, especially 50/60 Hz noise from electrical power lines that can be picked up by the patient's body. As shown in FIG. 3, the DRL circuit uses buffered Fz′ (BUF_Fz′) reference signal to generate reactive guard for the common signal of the electrodes. In some embodiments, the DRL circuit makes the system CMRR nearly flat within the EEG frequency range. Further, in some embodiments, the buffered Fz′ (BUF_Fz′) is used for active guarding of the shielded electrode leads. The active guarding helps prevent low frequency environmental noises from being inductively coupled to the signal inputs in the electrode wires. Active guarding is used either as a standalone noise-control measure, or in conjunction with signal amplification before passing the signal into the electrode wires.

In some exemplary embodiments, a two-stage RF filter is added to improve electromagnetic interference, particularly for radiated RF in the frequency range of 80 MHz to 2.4 GHz. As shown in FIG. 2, the first set of RF filters FT1, FT2, . . . , FT8 (corresponding to electrodes F1, F2, F7, F8, Fz′, Fpz, A1 and A2) are placed in close proximity to the electrodes, and are referenced to RF ground (RF_GND). The first RF filter comprises a feed-through high-Q RF capacitor tuned to the critical RF frequency range of 800 MHz to 1800 MHz. The second set of RF filters comprises common mode filters (for example, R3/C3, R12/C12 for the channel CH6) at the input of instrumentation amplifiers 30. The second stage RF filters are referenced to analog/power ground (A_GND). These filters are tuned to the lower end of the RF frequency range. To attenuate common-mode interference in the differential signal, the common mode RF filters are made symmetrical using matched resistors and capacitors.

Further, in some illustrative embodiments, a Faraday cage is formed around the plastic encapsulating box of the patient sensor 40 in order to shield the circuitry from electromagnetic interference. The cage is electrically connected to the RF ground (RF_GND). The shield of electrical signal cables linking the patient sensor and the base unit are also connected to RF_GND. In some embodiments, the shield of the outgoing signal cables are connected to the power ground in the base unit 42 at the point of lowest impedance. Care is taken to not locally interconnect the RF ground and power ground, to prevent creation of ground loops and ground currents.

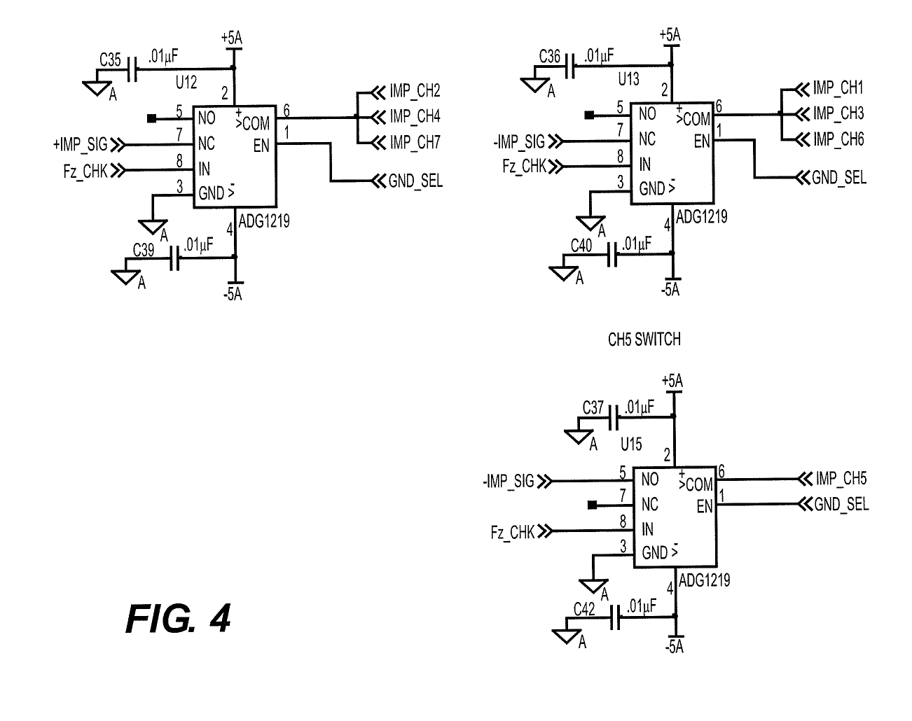

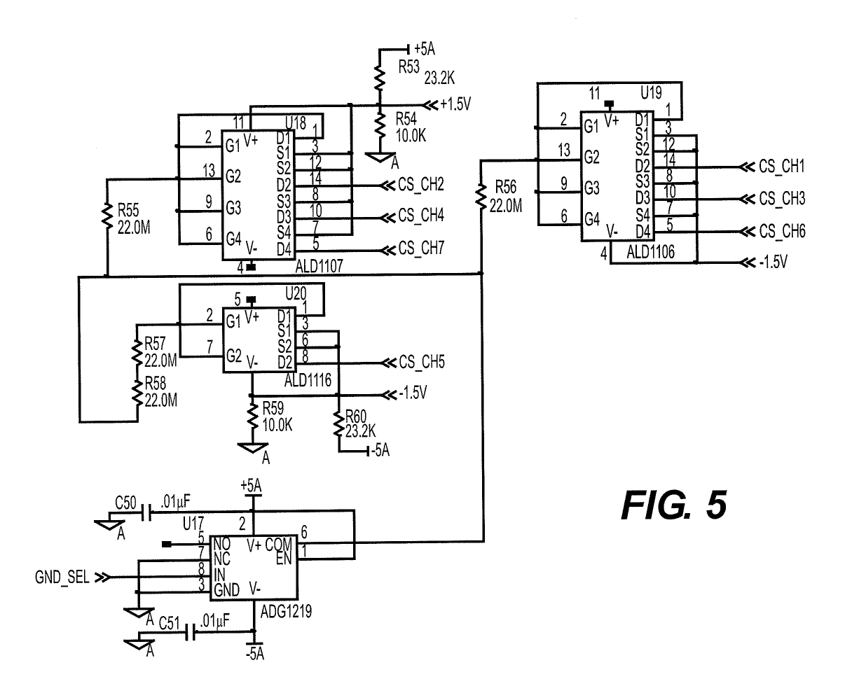

In certain embodiments, resistive AC current generators (for example, resistors R1, R5, R11, R18, R22, R27 and R31, as shown in FIG. 2) are used for the measurement of the electrode contact impedance on the skin prior to signal acquisition. The voltage source used for the impedance measurement is a 0.25 V p-p SINE and -SINE of approximately 37 Hz. Simultaneous application of both polarities helps to balance the head common voltage by keeping the return current (through Fpz electrode) near zero while the other electrodes are applied. In one embodiment, the impedance is measured by applying the opposite polarity signals to two groups of three electrodes through switches U12 and U13, as shown in FIG. 4. The impedance for channel 5 is measured separately through a switch U14, also illustrated in FIG. 4. AC impedance measurement is done in two phases. In the first phase, voltage is applied to all electrodes except Fz′ electrode (Fz′ voltage is equal to the head common). Since the original Fz′ signal is a common reference for every single electrode, a particular impedance is differentially represented at the IA's output as the voltage drop across the source impedance (measuring current is nearly constant). In the second phase, the only electrode to which voltage is applied is Fz′. In this case, the voltage drop across Fz′ and Fpz is observed at the output of the Fz′ instrumentation amplifier (the input of that IA is referenced to Fpz and the current is the same as in first phase). During data collection, the AC excitation is switched off, and small DC currents are applied to check the electrode contact quality. The DC impedance measurement provides continuous monitoring of electrode contact quality during data collection. After the initial electrode impedance measurement, the DC offset voltages resulting from the DC current flowing through the electrodes are recorded as referential values, and compared to the initial measurement values in real-time. If the difference between the two measurements is greater than a preset level set by the user, an alarm is generated to alert the user, who can choose to stop the data collection. In some illustrative embodiments, the DC currents are generated by matched low-leakage MOSFETS using switches U18, U19 and U20, as shown in FIG. 5. The current value is defined by reference MOSFET G-S voltage difference of 0.5 V and 22M ohm resistors (R55, R56 and R57/R58). The switch U20 is used to switch off all current sources during AC impedance measurement.

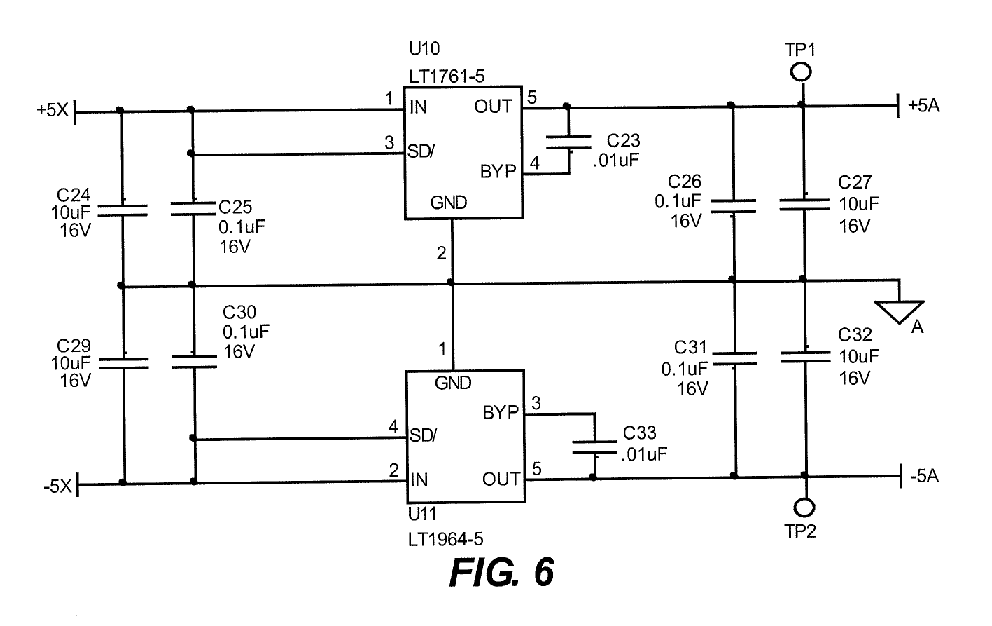

The power for the circuitry in patient sensor 40 is provided through low-noise power supplies. In exemplary embodiments, the circuitry of the patient sensor 40 is powered by low noise, low dropout linear regulators (LDOs), such as LT1761. The power supply voltages are +5V(+5 A) and −5V(−5 A) delivered from unregulated voltages (+/−6V) through LDOs U10 and U11, as illustrated in FIG. 6.

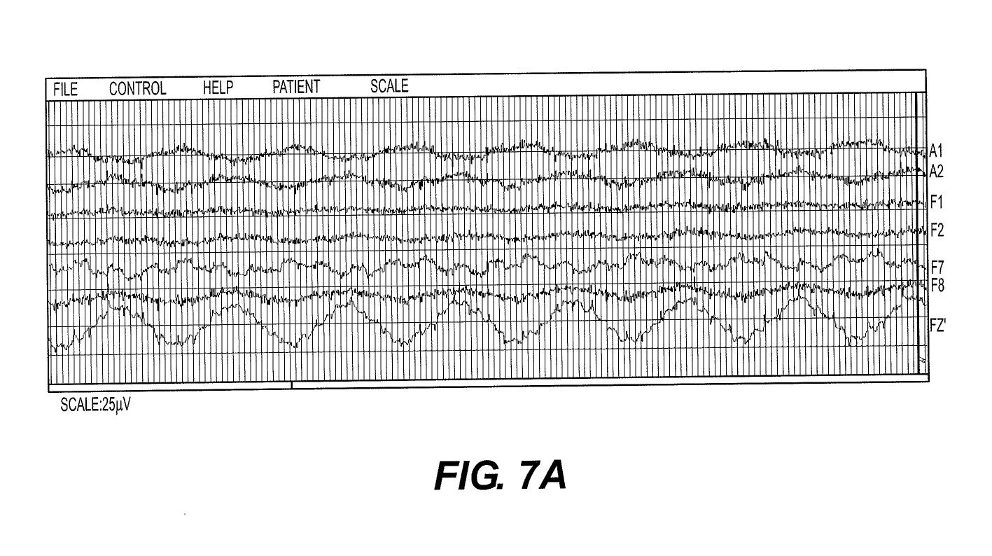

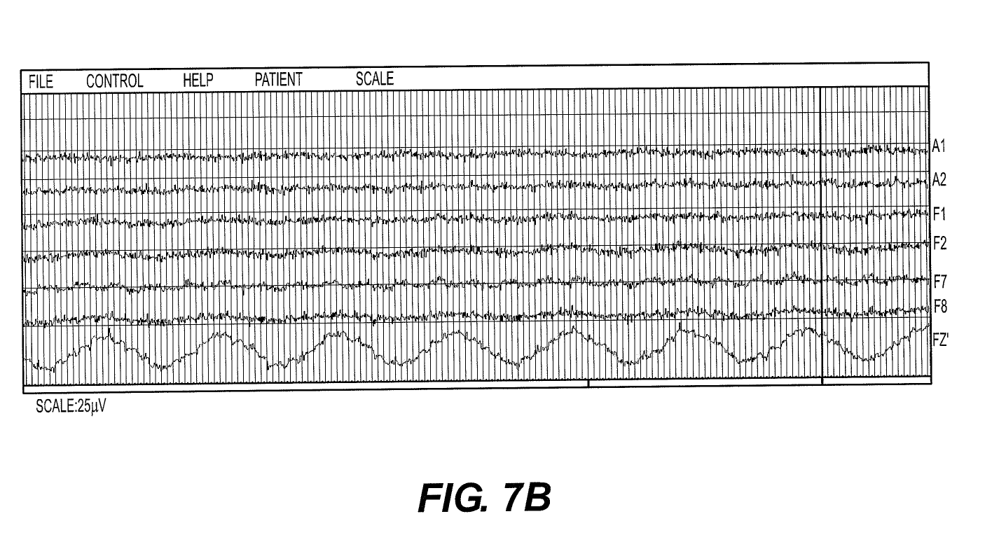

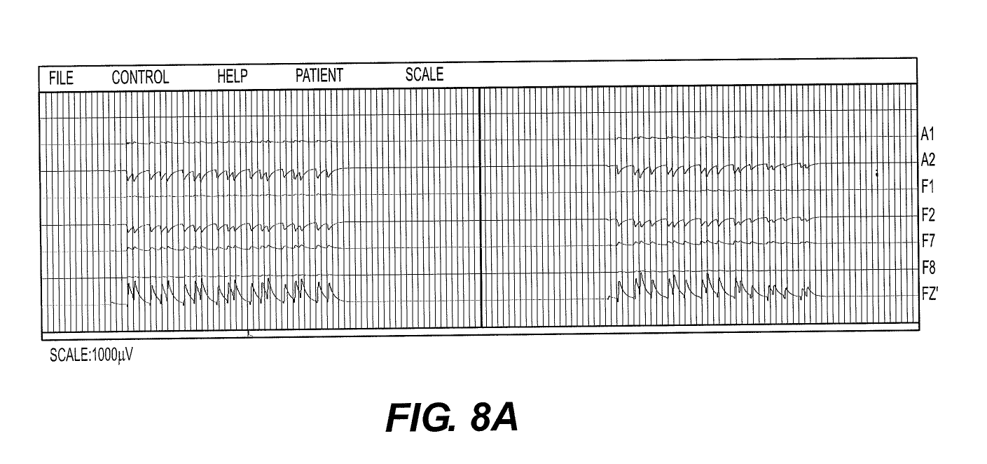

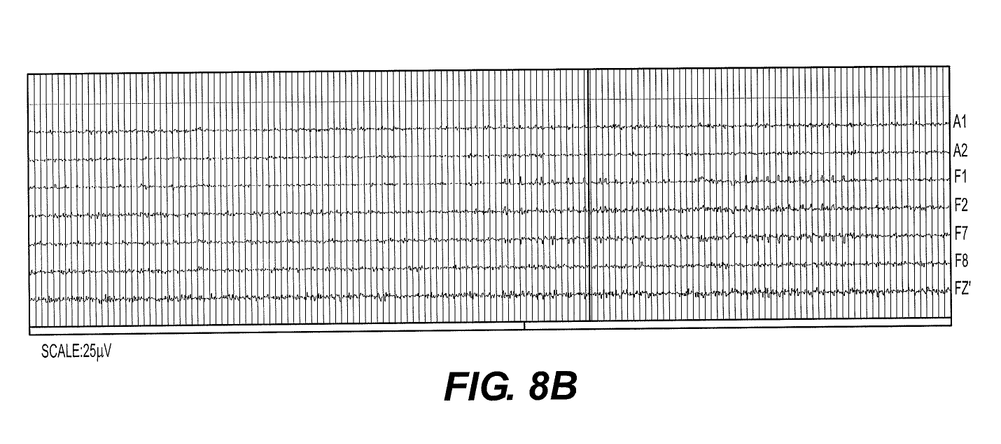

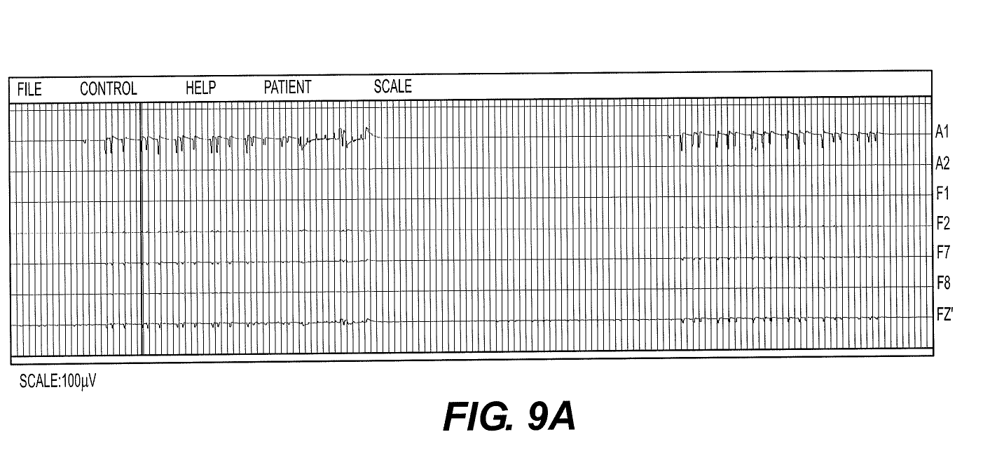

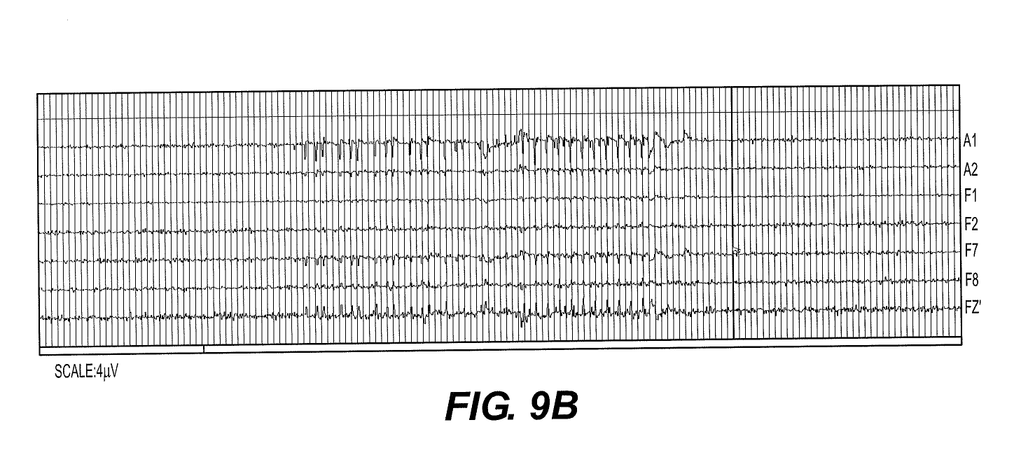

The brain electrical signals acquired by electrodes 20 are transmitted through the differential electrode channels (CH1, CH2, . . . , CHn) to the base unit 42 for further processing. The noise protection circuitry of patient sensor 40, as described in the present disclosure, reduces noise contamination of the signals during the transmission. FIG. 7A shows brain electrical signals that were transmitted to base unit 42 through standard electrode cables, and FIG. 7B illustrates brain electrical signals that were acquired and transmitted through patient sensor 40. As clearly seen in the figures, the signals transmitted using patient sensor 40 have significantly lower environmental noise effects. FIGS. 8A and 8B illustrate brain electrical signals that were exposed to 900 MHz, cellular telephone pulsed electromagnetic field. The signals shown in FIG. 8A were collected through standard electrode channels, and the signals in FIG. 8B were acquired and transmitted through patient sensor 40. As shown in the figures, the signals transmitted using patient sensor 40 have lower electromagnetic interference as compared to the signals that were transmitted to the base unit through standard electrode cables. Similarly, FIGS. 9A and 9B illustrate brain electrical signals that were exposed to 1800 MHz RF electromagnetic field. The signals shown in FIG. 9A were collected through standard electrode channels, and the signals in FIG. 9B were acquired and transmitted using patient sensor 40. As depicted in FIG. 9B, the signals transmitted using patient sensor 40 were protected from the RF emissions, and the recorded signals were visibly cleaner and had higher signal-to-noise ratio.

In some embodiments, the patient sensor 40 includes an analog-to-digital converter (ADC) to digitize the acquired brain electrical signals prior to receipt by the base unit 42. The digital electronics module 50 in the base unit 42 then processes the digitized data acquired from the patient sensor 40 to aid in interpretation of data pertaining to brain electrical activity. Further, as shown in FIG. 1A, the digital electronics module 50 may be operatively connected with a number of additional device components.

In some embodiments, base unit 42 comprises non-linear adaptive electronic systems, such as non-linear amplifiers, which assist in the processing of high-frequency weak brain signals acquired in extremely noisy environments. The non-linear amplifier systems utilize either a non-linear scale for compression of the dynamic range (such as logarithmic) or a closed loop system to remove reference common mode noise from individual or groups of electrode channels from which the measurement is taken.

Referring again to FIG. 1A, the base unit 42 may include a display 44, which can be a LCD screen, and can further have a user interface 46, which can be a traditional keyboard-type interface. In some embodiments, display 44 and user interface 46 are integrated into a graphical touch screen interface for entering user input and displaying test results. In some illustrative embodiments, a multi-channel input/output interface is provided between the patient sensor 40 and the base unit 42 to facilitate bidirectional communication of signals to and from the processor 51, such that, for example, a command from the user entered through the user interface 46 can start the signal acquisition process of the patient sensor 40. The input/output interface may include permanently attached or detachable cables or wires, or may include a wireless transceiver, capable of wirelessly transmitting and receiving signals to and from the patient sensor and the base unit. In one embodiment, patient sensor 40 includes two reusable patient interface cables which are designed to plug into the base unit 42 and provide direct communication between the patient sensor 40 and the base unit 42. The first cable is an electrical signal cable 41a, which delivers the acquired brain electrical signals to the base unit 42 for signal processing. The second cable is a stimulus cable 41b, which delivers stimuli to the patient during a neurological evaluation. Base unit 42 may include a stimulus generator 54 for providing the neurological stimuli to the patient during an evaluation. In one embodiment, stimulus cable 41b connects stimulus generator 54 to earphone 35 on patient sensor 40 for delivering auditory stimulus to generate Auditory Evoked Potential (AEP). Other auditory stimuli may also be used, to evoke mid-latency (20-80 milliseconds) or late auditory responses (>80 milliseconds), including the P300. Stimulus cable 41b may also be used to deliver other sensory stimuli to a patient, for example, visual or tactile stimuli, to elicit evoked potential response during a neurological evaluation. In some embodiments, small electrical signals are applied close to the nerves of the peripheral nervous system of a patient to elicit somatosensory evoked potentials (SSEP). The processor 51 is configured to denoise and process both the spontaneous brain electrical signals as well as evoked potentials generated in response to the applied stimuli. All the noise removal techniques described herein are also effective for removing noise in the electrodes and electrode cables used for the application of the evoked potential stimuli.

In an exemplary embodiment, noise artifacts are removed from the acquired signal in the signal processor 51, and the denoised signal is then processed to extract signal features and classify the extracted features according to instructions loaded into memory 52, as set forth in commonly assigned U.S. patent application Ser. Nos. 11/195,001, 12/041,106 and 12/639,357, which are incorporated herein by reference in their entirety. The memory 52 may further contain interactive instructions for using and operating the device to be displayed on the screen 44. The instructions may comprise an interactive feature-rich presentation including a multimedia recording providing audio/video instructions for operating the neuro-assessment apparatus 10, or alternatively simple text, displayed on the screen, illustrating step-by-step instructions for operating and using the apparatus. The inclusion of interactive instructions with the apparatus eliminates the need for extensive user training, allowing for deployment and uses by persons other than medical professionals. The memory 52 may also contain a reference database, including collected population data or data indicative of the individual baseline that may be used for data processing. In an exemplary embodiment, a reference database may be accessed from a remote storage device via a wireless or a wired connection. Similarly, data collected from the subject by the neuro-assessment apparatus 10 may be recorded in the database for future reference.

The results from the processor 51 may be displayed on the display 44, or may be saved in external memory or data storage device 47, or may be displayed on a PC 48 connected to the base unit 42. In one embodiment, base unit 42 contains a wireless power amplifier coupled to an antenna to transmit the results wirelessly to a remote network or PC 48 or the external memory 47 to store the results. In yet another embodiment, the results are transmitted wirelessly or via a cable to a printer 49 that prints the results. Further, in some embodiments, base unit 42 contains an internal rechargeable battery 43 that can be charged during or in between uses by battery charger 39 connected to an AC outlet 37. The battery may also be charged wirelessly through electromagnetic coupling by methods known in the prior art.

In another embodiment, the processor 51 transmits a raw, unprocessed signal acquired from a patient to the computer 48. The computer then performs the denoising process, analyzes the signal, extracts signal features, classifies the features and outputs the results. The unprocessed brain electrical signals recorded from a patient may also be stored in an external memory device for future reference and/or additional signal processing.

In one exemplary embodiment, the patient sensor 40 and the base unit 42 along with the charger 39 may come as a kit for field use or point-of-care applications. In yet another embodiment, both the patient sensor 40 and the base unit 42 may be configured to reside on a common platform, such as a headband, to be attached to a patient's head.

The neuro-assessment apparatus 10 is designed for near-patient testing in emergency rooms, ambulatory setting, and other field applications. The neuro-assessment apparatus is intended to be used in conjunction with CT scan, MRI, fMRI or other imaging studies to provide complementary or corroborative information about a patient's neurological condition. The key objective of point-of-care neuro-assessment is to provide fast results indicating the severity of a patient's neurological condition, so that appropriate treatment can be quickly provided, leading to an improved overall clinical outcome. For example, the neuro-assessment apparatus 10 may be used by an EMT, ER nurse, or any other medical professional during an initial patient processing in the ER or ambulatory setting, which will assist in identifying the patients with emergency neurological conditions. It will also help ER physicians in corroborating an immediate course of action, prioritizing patients for imaging, or determining if immediate referral to a neurologist or neurosurgeon is required. This in turn will also enable ER personnel to optimize the utilization of resources (e.g., physicians' time, use of imaging tests, neuro consults, etc.) in order to provide safe and immediate care to all patients.

In addition, the neuro-assessment apparatus 10 is designed to be field-portable, that is, it can be used in locations far removed from a full-service clinic—for example, in remote battlefield situations distant from military healthcare systems, during sporting events for indentifying if an injured athlete should be transported for emergency treatment, at a scene of mass casualty to identify patients who need critical attention and immediate transport to a hospital, or at any other remote location where there is limited access to well-trained medical technicians.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A device for acquiring and processing brain electrical signals from a patient, comprising:
a patient sensor adapted for attachment to the patient's head, the patient sensor comprising:
at least one active electrode and at least one reference electrode; and
a pre-amplification circuit positioned in close proximity to the at least one active electrode and at least one reference electrode, the pre-amplification circuit comprising:
at least one differential amplifier, wherein the at least one differential amplifier receives input signals from the at least one active electrode and the at least one reference electrode; and
two-stage RF filters comprising feed-through RF capacitors and symmetrical common mode filters;
a handheld base unit comprising a signal processor, the base unit being operatively coupled to the patient sensor for processing the brain electrical signals.

2. The device of claim 1, wherein the patient sensor further comprises a modified Driven Right Leg circuit to generate reactive guard for the common signal of the at least one active electrode and the at least one reference electrode.

3. The device of claim 1, wherein the patient sensor further comprises a Faraday cage assembly to provide RF shielding.

4. The device of claim 1, wherein the patient sensor further comprises AC current generating resistors for impedance measurements.

5. The device of claim 1, wherein the patient sensor further comprises DC current-generating MOSFETs for resistance checking.

6. The device of claim 1, wherein the patient sensor further comprises low dropout, low noise linear regulators for providing power.

7. The device of claim 1, wherein the at least one differential amplifier is a high input impedance instrumentation amplifier.

8. The device of claim 1, wherein the active electrode is configured to acquire both spontaneous and evoked potentials.

9. The device of claim 1, wherein the base unit further comprises at least one non-linear amplifier.

10. The device of claim 1, wherein the base unit further comprises a stimulus generator.

11. The device of claim 1, wherein the at least one active electrode and at least one reference electrode are each freely arranged on the patient's head.

12. The device of claim 1, wherein the at least one active electrode and at least one reference electrode are each arranged on a headgear.

13. A method of reducing noise contamination during acquisition of bioelectric signals from a patient, comprising the steps of:
providing a patient sensor comprising a pre-amplification circuit, the pre-amplification circuit comprising at least one active electrode channel, at least one reference electrode channel and at least one differential amplifier;
providing two-stage RF filters on the at least one active electrode channel and the at least one reference electrode channel;
providing a modified Driven Right Leg circuit to generate reactive guard for the common signal of the at least one active electrode and the at least one reference electrode;
providing a Faraday cage assembly within the patient sensor to provide RF shielding; and
making differential measurements of the input bioelectric signals from the at least one active electrode channel and the at least one reference electrode channel.

14. The method of claim 13, wherein the two-stage RF filters comprise feed-through RF capacitors and symmetrical common mode filters.

15. The method of claim 14, wherein the common mode filters are made symmetrical by using matched resistors and capacitors.

16. The method of claim 13, further comprising the step of making impedance measurements to determine the quality of electrode contact on the skin of the patient.

17. The method of claim 16, wherein the impedance measurement is made using AC current generated resistors.

18. The method of claim 13, further comprising the step of resistance checking to determine the quality of electrode contact on the skin of the patient.

19. The method of claim 18, wherein the resistance checking is made using DC current-generating MOSFETs.

20. The method of claim 13, wherein the at least one differential amplifier is a high input impedance instrumentation amplifier.

21. The method of claim 13, wherein power for the patient sensor is provided through a low noise power supply.

22. The method of claim 21, wherein the low noise power supply comprises low dropout linear regulators.

23. The method of claim 13, wherein the at least one active electrode channel and the at least on reference channel are connected to shielded electrode connectors.

24. The method of claim 13, wherein the bioelectric signal comprises brain electrical signals.

25. The method of claim 24, wherein the brain electrical signals comprise spontaneous and evoked potentials.

26. The method of claim 13, wherein the patient sensor comprises at least one active electrode and at least one reference electrode each for attachment to the patient's forehead.

27. The method of claim 13, further comprising the step of transmitting low impedance output signals through shielded cables to a handheld base unit operatively connected to the patient sensor.

28. The method of claim 27, wherein the bioelectric signals are amplified further in the base unit prior to signal processing.

29. The method of claim 28, wherein the base unit comprises one or more non-linear amplifiers to amplify the bioelectric signal.

30. The method of claim 27, wherein the base unit comprises a signal processor for processing the bioelectric signals.
